(19) United States
(12) Patent Application Publication
(10) Pub. No.: US 2024/0425461 A1
(43) Pub. Date: Dec. 26, 2024

(54) PREPARATION PROCESS OF INTERMEDIATE, SYNTHESIS PROCESS OF AMINO IMIDAZOLE CARBOXYLATE ESTER AND HPLC PURITY MEASURE METHOD

(71) Applicant: ASCENTAWITS PHARMACEUTICALS, LTD., Shenzhen (CN)

(72) Inventors: Jianxin Duan, Shenzhen (CN); Zhaoqiang Lu, Shenzhen (CN); Jinwei Fan, Shenzhen (CN); Qunhui Huang, Shenzhen (CN); Yanjun Zhang, Shenzhen (CN); Weisu Chen, Shenzhen (CN); Wei Qin, Shenzhen (CN)

(21) Appl. No.: 18/209,306

(22) Filed: Jun. 13, 2023

(51) Int. Cl. C07D 233/90; C07C 227/16

(52) U.S. Cl. CPC C07D 233/90; C07C 227/16

(57) ABSTRACT

This invention relates to a process for preparing an intermediate of aminoimidazole carboxylate, a process for synthesizing aminoimidazole carboxylate and an HPLC method for determining purity. The process of preparing the intermediate of aminoimidazole carboxylate comprises subjecting a compound of formula I-2 and a formate as the reactants to a first-step reaction in a benzene solvent and at a temperature of −5° C.−5° C., with the addition of a solution of sodium alkoxide in such a way that the temperature of the reaction solution is not higher than 5° C., to obtain the intermediate of aminoimidazole carboxylate or a mixture comprising a compound of formula I-3, i.e., the intermediate of aminoimidazole carboxylate. By using a sodium alkoxide reagent, as a comparatively safe alternative to NaH and potassium tert-butoxide which are prone to cause fire hazards or explosions and are not suitable for use in large-scale production, the process of the present application can ensure smooth progression of the reaction to the product and effectively improve the overall safety of the reaction without noticeably impairing the reaction efficiency.

### TECHNICAL FIELD

The present application relates to the technical field of preparing an intermediate of aminoimidazole carboxylate, and synthesizing and detecting aminoimidazole carboxylate, and in particular to a process for preparing an intermediate of aminoimidazole carboxylate, and a process for synthesizing aminoimidazole carboxylate and a method for detecting the same.

### BACKGROUND ART

TH-302 (Evofosfamide, CAS No.: 918633-87-1) is a 2-nitroimidazole-triggered Hypoxia-Activated Prodrug (HAP) of bromo-isophosphoramide. Under hypoxic conditions, inactive TH-302 prodrug can release bromo isophosphoramide mustard (Br-IPM) that is highly toxic. TH-302 exhibits broad-spectrum biological activity in vivo and in vitro and specific hypoxia-selective activation activity, and induces H2AX phosphorylation and has a DNA cross-linking activity, thereby leading to cell cycle arrest. Therefore, this compound has been developed as potential anticancer drugs by many pharmaceutical companies and scientific research institutes.

Research articles published by Meng F (Meng Fanying) et al. have disclosed that TH-302 has broad-spectrum activity against various tumors, and has excellent hypoxia-selective activity-enhancing effect. Studies have shown that the cytotoxicity in vitro of TH-302 in 32 human cancer cell lines under hypoxic conditions is significantly higher than that under normoxic conditions, suggesting that the compound has selective cytotoxicity in cancer cells under hypoxic conditions. The use of human cells over-expressed by one-electron reductase (POR) confirmed the mechanism of enhanced one-electron reductase-dependent activity of TH-302 under hypoxic conditions, as shown in the following Reaction Scheme 1:

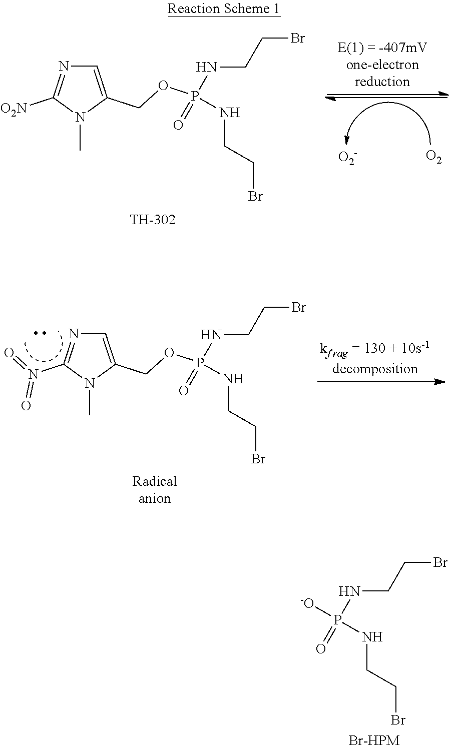

The TH-302 prodrug is reduced by cytochrome P450 oxidoreductase to give free radical anion as an intermediate. Owing to its instability, the free radical anion is then decomposed into the cytotoxin Br-IPM with cytotoxicity, which exerts its efficacy by cross-linking with the DNA chain: mainly inactivating DNA by cross-linking with it via alkylation at the 7-position in the guanine structure on DNA, thereby making it being incapable of normal replication and being possible to achieve the effect of inhibiting proliferation pf (cancer) cells.

Further in-vivo animal model experiments have showed that TH-302 can effectively reduce hypoxic regions in various tumors (Sun, J. D., Liu, Q., Wang, J., Ahluwalia, D., Ferraro, D., Wang, Y., Duan, J. X., Ammons, W. S., Curd, J. G., Matteucci, M. D., & Hart, C. P. (2012). Selective tumor hypoxia targeting by hypoxia-activated prodrug TH-302 inhibits tumor growth in preclinical models of cancer. Clinical cancer research: an official journal of the American Association for Cancer Research, 18 (3), 758-770. https://doi.org/10.1158/1078-0432.CCR-11-1980), and result in increased immunotherapeutic efficacy (Jayaprakash, P., Ai, M., Liu, A., Budhani, P., Bartkowiak, T., Sheng, J., Ager, C., Nicholas, C., Jaiswal, A. R., Sun, Y., Shah, K., Balasubramanyam, S., Li, N., Wang, G., Ning, J., Zal, A., Zal, T., & Curran, M. A. (2018). Targeted hypoxia reduction restores T cell infiltration and sensitizes prostate cancer to immunotherapy. The Journal of clinical investigation, 128 (11), 5137-5149. https://doi.org/10.1172/JCI96268).

The above findings have indicated that TH-302 still has the potential to be used in the development of anticancer drugs, and thus there still remains a need to develop a highly-efficient process for preparing or synthesizing TH-302.

The synthesis of TH-302 was first disclosed by Duan Jianxin (J-X. Duan) in the patent application PCT/US2006/025881, or WO2007/002931 filed by Threshold Company, and the counterpart Chinese patent application with the Publication No. CN101501054A. Particularly, in its Examples 22 to 24, the synthesis routes and preparation process (hereinafter referred to as the “Threshold's method”) of a key intermediate, 1-N-methyl-2-aminoimidazole-5-carboxylate have been described. The specific routes are shown below:

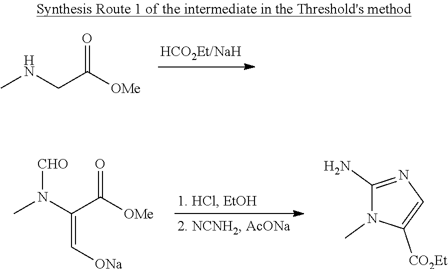

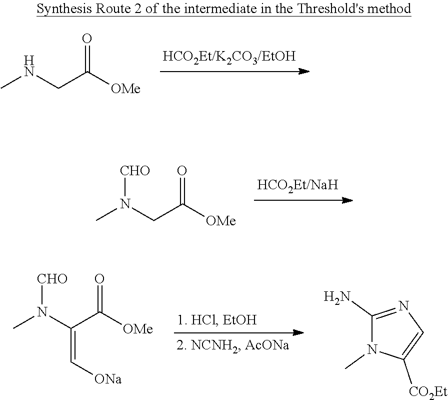

Particularly, in the Routes 1 and 2, sarcosinate methyl ester hydrochloride was used as the starting material and reacted with NaH in a one-step reaction or with K₂CO₃ and NaH in a two-step reaction to obtain the intermediate,

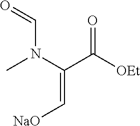

which was hydrolyzed with hydrochloric acid under heat conditions; and the hydrolylate was cyclized under heat conditions and with the addition of sodium acetate and cyanamide to give 1-N-methyl-2-aminoimidazole-5-carboxylate.

Starting from this route, Liam J. O'Connor et al. (Efficient synthesis of 2-nitroimidazole derivatives and the bioreductive clinical candidate Evofosfamide (TH-302), Org Chem Front, 2015, 2, 1026-1029) have improved the Threshold's method (hereinafter referred to as the “Liam's improvement”):

The first improvement is to change the conditions for cyclization reaction under heat conditions and with the addition of sodium acetate and cyanamide in the Threshold's route to obtain 1-N-methyl-2-aminoimidazole-5-carboxylate: particularly, with respect to the oily substance obtained after hydrolysis with hydrochloric acid under heat conditions, the original operation of adding it to an aqueous acetic acid, sodium acetate and cyanamide, and then reacting for 1 hour under stirring at 95° C. were changed to a new operation of adding it to an aqueous solution of 70% ethanol and cyanamide, and then reacting for 1.5 hours under stirring at 100° C.:

The second improvement is to replace methyl sarcosinate hydrochloride as the starting material, with ethyl sarcosine hydrochloride.

With the above-mentioned improvements, the yield of the synthesized ethyl 1-N-methyl-2-amino-imidazole-5-carboxylate was increased to 48-54%, than 10-25% in the Threshold's method.

In both the Threshold's method and Liam's improvement, NaH was used to prepare the intermediate

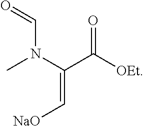

Due to the nature of NaH and the characteristics of the reaction, however, the reaction system must be placed in a low-temperature environment during the preparation process, and kerosene or THF suspension of NaH has to be added separately, and the resulting solution must be reacted under a protective atmosphere such as nitrogen gas. More importantly, in the environment of a chemical plant, NaH owing to its high chemical reactivity can react with oxygen, carbon dioxide, water vapor, acid gas, or other substances that are present in the air, thereby releasing heat and hydrogen to cause risks of combustion and explosion. Thus, NaH as the reagent in said reaction may impose great safety hazards when it is applied in a factory for large-scale production.

### SUMMARY OF THE INVENTION

In view of the above concerns, the present application provides an intermediate of aminoimidazole carboxylate and a process for synthesizing the aminoimidazole carboxylate, which can effectively improve the safety of the reaction without impairing the reaction efficiency.

It has been recognized by those skilled in the art that in the relevant technology, due to the use of highly active reagents such as NaH, the reaction duration is relatively short, and generally the conversion can be completed within a time period of 8 hours or less. However, there are great safety hazards in the reaction process. NaH with high chemical reactivity can react with oxygen, carbon dioxide, water vapor, acid gas or other substances that are present in the air, thereby releasing heat and hydrogen to cause risks of combustion and explosion.

In order to eliminate the safety hazards of the above reaction, those skilled in the art have made attempts to use reagents that enable mild reactions. Nonetheless, it should take note that reagents that allow relatively mild reactions will inevitably hinder progression of the reaction, such as a drastic reduction in conversion or yield, thus resulting in incomplete reaction.

For this reason, it has always been difficult for those skilled in the art to find suitable mild reagents without compromising the thoroughness of the reaction.

In a first aspect, the present application provides a process for preparing an intermediate of aminoimidazole carboxylate, comprising subjecting a compound of formula I-2 and a formate as the reactants to a first-step reaction in a benzene solvent and at a reaction temperature of from −5° C. to 5° C., with the addition of a solution of sodium alkoxide in such a way that the temperature of the reaction solution is not higher than 5° C., to obtain the intermediate of aminoimidazole carboxylate or a mixture comprising a compound of formula I-3, i.e., the intermediate of aminoimidazole carboxylate:
wherein the compounds of formulae I-2 and I-3 have the following structures, respectively:

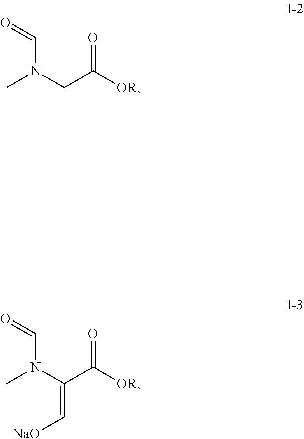

wherein the R group is a substituted or unsubstituted C₁-C₆ alkyl group.

Regarding the temperature of the above-mentioned first-step reaction, the reason why this application adopts the specific temperature range resides in that: the sodium alkoxide that is added in the reaction is a strong base; if the temperature of reaction is too high, the carboxylate would be hydrolyzed by sodium alkoxide. Thus, the temperature of this reaction cannot be too high, and accordingly the alcohol solution of sodium alkoxide cannot be added too rapidly to cause excessively high local temperature and to form by-products from the hydrolysis of the carboxylate. In particular, the present invention requires the controlled rate of addition and thorough stirring to ensure that the temperature during the addition would not be higher than 5° C.: besides, after the addition, the reaction system should be kept under stirring at a temperature of from −5 to 5° C. for the duration of 8 hours or longer until the reaction is complete. If the reaction temperature exceeds 5° C., it may lead to the formation of by-products at a great amount from the hydrolysis of the carboxylate, and if the reaction temperature is too low (less than −5° C.), it may lead to undesirably long reaction duration and other side reactions. Therefore, the temperature of −5-5° C. and the duration of 8 hours or longer are the optimal reaction conditions with comprehensive considerations of various factors.

Optionally, the benzene solvent is selected from C₇-C₉ liquid-state substituted benzenes, benzene or a mixture thereof, preferably selected from toluene, xylene, trimethylbenzene, ethylbenzene, propylbenzene, isopropylbenzene or a mixture thereof:
the formate is selected from methyl formate, ethyl formate, n-propyl formate, isopropyl formate or a mixture thereof;
the sodium alkoxide is selected from sodium methylate, sodium ethylate, sodium n-propoxide, or sodium isopropoxide;
in the compounds of formulae I-2 and I-3, the R group is a C₁-C₃ alkyl group, preferably ethyl; the reaction temperature of the first-step reaction is ranging from −5 to 0° C.;
the reaction time of the first-step reaction is 8 to 16 hours;
in the first-step reaction, the volumetric mass ratio of the benzene solvent to the compound of formula I-2 is (1.40 m1-1.50 m1): 1 g.

Optionally, the mass ratio of the compound of formula I-2 to the sodium alkoxide is less than or equal to 1, preferably in the range of from 0.80 to 1;
the mass ratio of the compound of formula I-2 to the formate is less than or equal to 0.5, preferably in the range of from 0.4 to 0.5.

Optionally, the sodium alkoxide is added at a temperature of lower than 0° C.

Specifically, according to the first aspect of the present application, the compound of formula I-2 in a mass amount of ml is added to toluene in a volume of v, and then ethyl formate in a mass amount of m2 is added; the reaction solution is then cooled to a temperature ranging from −5 to 0° C.; and an ethanol solution containing sodium ethoxide in a mass amount of m3 is added batchwise at a controlled rate so that the temperature of the reaction solution does not exceed 5° C. Once the addition of the ethanol solution has been completed, the resulting solution is reacted at a controlled temperature ranging from −5 to 5° C. for 8-16 hours, wherein the R group in the compound of formula I-2 is ethyl.

Optionally, the v: ml is 1.42 ml: 1 g; m2; ml is 1.00; and m3: ml is 0.50.

Optionally, the process further comprises the following steps:
adding, to the mixture obtained in the first-step reaction, a weak polar solvent in a volume 3-5 times as much as that of the mixture; after stratification, removing the upper layer to leave the lower layer as a reactant for an acid hydrolysis step, wherein the weak polar solvent is petroleum ether, diethyl ether or n-hexane.

The use of the weak polar solvent here is intended to extract, from the reaction of converting the compound of I-2 to the compound of I-3, the solvent used (such as toluene), unreacted formate (such as ethyl formate) and possible by-products. Depending on the substances to be extracted, the weak polar solvent used here includes petroleum ether, diethyl ether, n-hexane, diphenyl ether, benzene, toluene, cyclohexane, hexane or kerosene, etc.

In a second aspect, the present application provides a process for synthesizing aminoimidazole carboxylate, which comprises the following steps:
preparing a compound of formula I-3, i.e., the intermediate of aminoimidazole carboxylate or a mixture comprising a compound of formula I-3, i.e., the intermediate of aminoimidazole carboxylate, according to the process for preparing the compound of formula I-3 as described above;
subjecting the compound of formula I-3 (i.e., the intermediate of aminoimidazole carboxylate) or a mixture comprising the compound of formula I-3 (i.e., the intermediate of aminoimidazole carboxylate) as the starting material, to acid hydrolysis to obtain a hydrolyzate of formula I-4;
wherein the hydrolyzate of formula I-4 is:

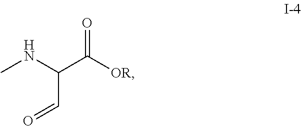

cyclizing the hydrolyzate of formula I-4 with cyanamide as reactant, to obtain a mixture comprising the compound of formula I, i.e., aminoimidazole carboxylate;
wherein the compound of formula I is:

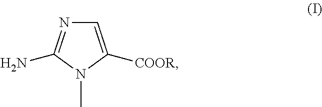

wherein the R group in the hydrolyzate of formula I-4 and in the compound of formula I is a C₁-C₆ alkyl group, preferably a C₁-C₃ alkyl group, more preferably an ethyl group.

Optionally, the acid hydrolysis specifically comprises the following steps:
adding under stirring, to the compound of formula I-3 (i.e., the intermediate of aminoimidazole carboxylate) or a mixture comprising the compound of formula I-3 (i.e., the intermediate of aminoimidazole carboxylate), ethanol or methanol in a volume 3-5 times as much as that of the compound or the mixture; adding concentrated hydrochloric acid; and then heating to a temperature ranging from 60-80° C., reacting under reflux for 0.5-2 hours and filtering;
concentrating the collected filtrate under reduced pressure at 40-45° C. until no dripping, so as to obtain the hydrolyzate of formula I-4;
the cyclization reaction specifically comprises the following steps:
mixing and dissolving the hydrolyzate of formula I-4 within an aqueous solution of cyanamide and an acetic acid solution of sodium acetate or a formic acid solution of sodium formate;
heating and reacting under reflux to obtain a mixture comprising the compound of formula I, i.e., the aminoimidazole carboxylate.

For the above-mentioned hydrolysis reaction, the acid used therein should be a non-oxidizing acid with strong concentration. Thus, nitric acid is not suitable. Suitable acids include concentrated hydrochloric acid and concentrated sulfuric acid. The acid should be in excess to ensure adequate and complete hydrolysis. Concentrated hydrochloric acid is convenient for the removal by neutralization in a next step, because the HCl in hydrochloric acid is a biogenic volatile gas, and it can be easily removed by evaporation under reduced pressure. Thus, the more preferableinorganic acid used herein is concentrated hydrochloric acid.

Optionally, the mass ratio of the cyanamide contained in the aqueous solution of cyanamide to the hydrolyzate of formula I-4 is 2:1:
the mass ratio of the sodium acetate or sodium formate to the hydrolyzate of formula I-4 is 2:1;
the volumetric mass ratio of the solvent acetic acid or formic acid to the hydrolyzate of formula I-4 is 10 ml: 1 g.

Optionally, after the cyclization reaction, the process further comprises the following steps: adjusting, with hydrochloric acid or sulfuric acid, the reaction mixture solution comprising the compound of formula I, i.e., the aminoimidazole carboxylate, obtained after the cyclization reaction, to a pH of 1, and then after thermal removal of acetic acid or formic acid, adjusting with a base to a pH of 8 to 9, filtering and stirring; and washing the filter cake with water to give a solid, which is dried to obtain the compound of formula I, i.e., the aminoimidazole carboxylate.

The base used here is simply intended to neutralize the acid by reacting with the added hydrochloric acid or sulfuric acid and to finally obtain a solution with a slightly alkaline pH value. Therefore, the base can be hydroxides of alkali metals such as NaOH and KOH, and of alkaline earth metals, or carbonates and bicarbonates of alkali metals such as Na₂CO₃, K₂CO₃, NaHCO₃, and KHCO₃.

In a third aspect, the present application provides an HPLC method for detecting the purity of ethyl 1-N-methyl-2-aminoimidazole-5-carboxylate, comprising subjecting a sample solution comprising ethyl 1-N-methyl-2-aminoimidazole-5-carboxylate to liquid chromatography under the following chromatographic conditions:
the separation column is a reverse-phase C₁₈ column;
the detection wavelength is 255 nm;
mobile phase A is 0.1% phosphoric acid solution, and mobile phase B is acetonitrile;
elution is carried out using a gradient elution procedure; and
the purity % of ethyl 1-N-methyl-2-aminoimidazole-5-carboxylate is calculated by using the area normalization method based on its peak area.

Optionally, the gradient elution procedure is:

| | Time/min. | | | | |
|---|---|---|---|---|---|
| | 0 | 30 | 35 | 35.1 | 40 |
| Mobile phase A % | 95 | 10 | 10 | 95 | 95 |
| Mobile phase B % | 5 | 90 | 90 | 5 | 5 |

wherein % is volume-based; and
the peak retention time of the ethyl 1-N-methyl-2-aminoimidazole-5-carboxylate is in the range of from 3 to 7 minutes.

The technical solutions provided by the present application bring about the following beneficial effects:
The present application uses relatively safe sodium alkoxide reagents (sodium methoxide, sodium ethoxide, and sodium propoxide) in replace of NaH that is prone to cause fire hazards and explosion and is not suitable for use on large-scale production;
By using sodium alkoxide as a nucleophilic reagent and benzenes as a solvent instead of the highly active reagents such as NaH, the present application can ensure smooth progression of the reaction to the desired product and effectively improve the overall safety of the reaction. Moreover, despite the mild reaction lasting for a prolonged period of time, the process of the present application can ensure complete reaction (with high yield or conversion), and thus can effectively improve overall safety of the reaction without noticeably impairing the reaction efficiency.

### BRIEF DESCRIPTION OF THE DRAWINGS

In order to illustrate the technical solutions described in the examples of the present application, the drawings to be mentioned in the examples will be introduced briefly below. Apparently, the drawings as described below only represent some examples of the present application; and as will be appreciated by those skilled in the art, other drawings can also be derived based on these drawings without exercising any inventive efforts.

FIG. 1 is the HPLC spectrogram of ethyl 1-N-methyl-2-aminoimidazole-5-carboxylate A4 obtained after recrystallization and purification of the kilogram-scale product of Example 4.

### DETAILED DESCRIPTION OF THE INVENTION

In order to more clearly explain its objects, technical solutions and advantages, the present invention will be further described in detail below in conjunction with the examples. It should be understood that the specific embodiments described herein are only for the purpose of illustrating the present disclosure and are not intended to limit the present disclosure in any respect.

### Example I

Ethyl aminoimidazole carboxylate is used as an example to illustrate the specific process.

Synthesis route of ethyl aminoimidazole carboxylate:

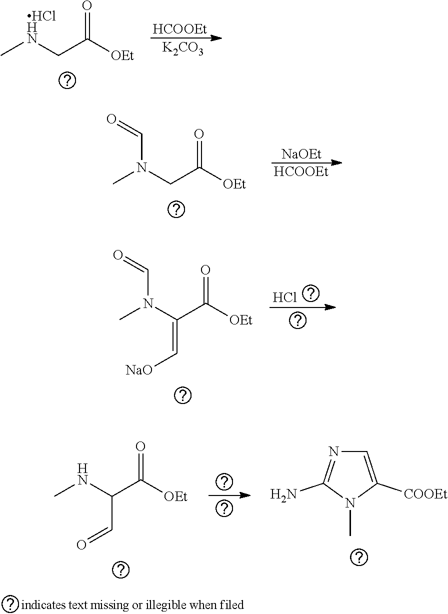

In the above route for the synthesis of ethyl aminoimidazole carboxylate A4, the intermediate A3 is depicted as an aldehyde structure

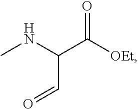

or can alternatively be as an enol structure

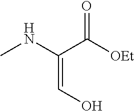

(Efficient synthesis of 2-nitroimidazole derivatives and the bioreductive clinical candidate Evofosfamide (TH-302), Org Chem Front, 2015, 2, 1026-1029). Although it can be represented by two different structures, the intermediate actually refers to the same substance.

The particular process is described as follows:
At room temperature and under the protection of nitrogen gas, 20.0 g of methylglycine ethyl ester hydrochloride A0, 76.4 g of ethyl formate, 20.0 g of anhydrous potassium carbonate, and 80 ml of 70% (V %) ethanol solution were added to a 500 ml three-necked flask. After the addition, the reaction mixture was heated to 55-90° C. and reacted under stirring with a magnetic stirrer. The reaction was considered complete when HPLC showed that the sample contained less than 1.0% of the starting material A0 (the reaction duration lasted for about 15 hours). Subsequently, the reaction mixture was cooled to 25-30° C. and filtered: the filtrate was collected and concentrated under reduced pressure at 40-45° C. until no dripping: the residue was extracted with ethyl acetate twice, 50 ml each time: the extract liquors were combined, dried with 10.0 g of anhydrous sodium sulfate for 3 hours and then filtered. The collected filtrate was concentrated under reduced pressure at 40-45° C. until no dripping, to give 16.5 g of A1.

16.5 g of A1 (about 0.11 mol) obtained in the previous step was added into 23.5 ml of toluene, to which then 16.5 g of ethyl formate (about 0.22 mol) was added. The reaction solution was cooled to a temperature within-5 to 5° C. An ethanol solution containing 8.32 g of sodium ethoxide (ethanol solution containing 8.32 g of sodium ethoxide, about 0.12 mol) was added batchwise at a controlled rate to keep the reaction solution below a temperature of 5° C. After the addition, the mixture solution was reacted for 16 hours with the temperature being controlled in the range of from −5 to 5° C., and then subjected to a post-treatment.

For the post-treatment, 165 ml of petroleum ether was added to the above reaction solution and stirred. The upper-layer supernatant was poured out, and the lower-layer solution was collected. The product A2 is exactly included in the lower-layer solution.

165 ml of ethanol was added to the lower-layer solution and stirred to obtain an orange-red solution; and 33 ml of concentrated hydrochloric acid was added. The reaction solution was heated to 60 to 80° C., and reacted under reflux for 1 hour. After completion of the reaction, the resulting solution was subjected to a post-treatment.

The reaction solution was cooled to 25-30° C. and filtered. The filtrate was collected and concentrated under reduced pressure at 40-45° C. to remove ethanol until no dripping, so as to obtain an oily substance containing the product A3.

The oily substance was cooled to 25-30° C., to which 165 ml of acetic acid, 8.6 g of cyanamide (50% aqueous solution, 0.20 mol), and 18.7 g of anhydrous sodium acetate (about 0.22 mol) were added. After addition, the reaction solution was heated to 90-100° C., reacted under reflux for 2 hours, and then subjected to a post-treatment.

The reaction solution was cooled to 20-25° C., adjusted with concentrated hydrochloric acid to a pH value of 1, and then concentrated under reduced pressure at 45-50° C. to remove acetic acid until no dripping. The resulting solution was adjusted with potassium carbonate to a pH value of 8-9, stirred for 1 hour and then filtered. The filter cake was washed with water twice to give a solid, which was vacuum-dried under reduced pressure at 40° C.-50° C. to obtain 6.68 g of the product A4.

HPLC showed that the above product of 6.68 g in weight has a purity of 82.21% and a yield of 30.32%, calculated using A0 as the starting material.

### Example II

The procedure in Example 1 was repeated, by using the same materials and operations. 7.94 g of the product A4 was obtained, with a purity of 92% and a yield of 36.04%, calculated using A0 as the starting material.

### Example III

This example describes an exploratory experiment in which the same process as Example 1 was repeated, and the reactants, reaction time, reaction temperature, and feeding ratio in the step of A1 to A2 were varied to explore their effects on the process of converting A0 to the final product A4. The feeding amount of A1 was fixed, to assess the purity and yield of the final product A4.

In all the reaction groups, the amount of A1 was fixed to 16.5 g; 100 g of A0 was used for preparation according to the above reaction process to give 83 g of A1, which was divided into 5 portions, three of which were added into each of the Reaction Groups Mar. 4, 2005. The Reaction Group 01 corresponds to the data from Example I, and the Reaction Group 02 corresponds to the data from Example II.

The yield is calculated based on A0; specifically, in the Reaction Groups Mar. 4, 2005, the yield is calculated based on 20 g of A0.

TABLE 1

| | | | Formate, | | | | |
|---|---|---|---|---|---|---|---|
| | Feeding | Benzene | mass or | Base, | Reaction | | Purity^{a} and |
| Reaction | amount | solvent, | molar | molar | temperature | | Yield^{b} of |
| group | of A1 | volume | amount | amount | and time | Post-treatment | A4 |
| 01 | 16.5 g, | Toluene, | Ethyl | Ethanol | 0-5° C., 16 h | Extract with | 82.21%, |
| | 0.11 mol | 23.5 ml | formate, | solution | | 165 ml of | 30.32% |
| | | | 0.22 mol | of sodium | | petroleum ether | |
| | | | | ethoxide, | | for | |
| | | | | 0.12 mol | | stratification, | |
| | | | | | | lower-layer | |
| 02 | 17.5 g, | Toluene, | Ethyl | Ethanol | 0-5° C., 16 h | Extract with | 92.00%, |
| | 0.11 mol | 23.5 ml | formate, | solution | | 165 ml of | 36.04% |
| | | | 0.24 mol | of sodium | | petroleum ether | |
| | | | | ethoxide, | | for | |
| | | | | 0.13 mol | | stratification, | |
| | | | | | | lower-layer | |
| 03 | 16.6 g, | Xylene | Ethyl | Methanol | 0-5° C., 16 h | Extract with | 85.65%, |
| | 0.11 mol | 23.5 ml | formate, | solution of | | 165 ml of | 34.20% |
| | | | 0.22 mol | sodium | | petroleum ether | |
| | | | | methoxide, | | for | |
| | | | | 0.12 mol | | stratification, | |
| | | | | | | lower-layer | |
| 04 | 16.6 g, | Ethylbenzene | Methyl | KOH + NaH, | 0-5° C., | Extract with | 74.23%, |
| | 0.11 mol | 23.5 ml | formate | 0.12 mol | 16 + 4 h | 165 ml of | 26.85% |
| | | | 0.22 mol | | | N-hexane | |
| | | | | | | for | |
| | | | | | | stratification, | |
| | | | | | | lower-layer | |
| 05 | 16.6 g, | Ethylbenzene | Methyl | KOH + potassium | 0-5° C., | Extract with | 68.5%, |
| | 0.11 mol | 23.5 ml | formate | tert-butoxide, | 16 + 2 h | 165 ml of | 31.28% |
| | | | 0.22 mol | 0.12 mol | | N-hexane | |
| | | | | | | for | |
| | | | | | | stratification, | |
| | | | | | | lower-layer | |

a is the purity calculated by the HPLC area normalization method;
b is the percentage yield calculated using A0 as the starting material by the formula: the mass of the final product/the mass of the theoretical product. Taking Group 1 as an example, 6.68 g/22.03 g=30.32%.

As can be seen from Table 1, the comparisons show that under the same conditions, the use of sodium methoxide and sodium ethoxide plays similar effect on the reaction, in terms of purity and yield.

In the Reaction Group 04 using KOH, almost no product was observed after adding 0.12 mol of KOH powder and carrying out the reaction at 0-5° C. for 16 hours. After adding 0.12 mol of NaH in kerosene dispersion, the reaction was completed in 4 hours. The same situation occurred in Reaction Group 05: almost no product was observed after adding 0.12 mol of KOH powder and carrying out the reaction at 0-5° C. for 16 hours. After adding 0.12 mol of potassium tert-butoxide powder, the reaction was completed in 2 hours.

By comparison, it can be seen that the conversion of A1 to A2 cannot be completed using hydroxides of alkali metal such as KOH and NaOH; although the use of reagents such as sodium methoxide and sodium ethoxide allows a slower reaction than that of NaH and potassium tert-butoxide, the conversion of A1 to A2 can be completed. And the yield is similar to those obtained when NaH or potassium tert-butoxide is used. Thus, it is feasible to use sodium methoxide and sodium ethoxide.

In addition, since sodium methoxide and sodium ethoxide are dissolved in methanol and ethanol and are supplied in the form of liquid solutions, the addition thereof can be carried out by using a dropping funnel. It can facilitate the controlled dropping time and dropping rate, and thereby the controlled reaction.

### Example IV

This example is amplification to large-scale from hectogram- to kilogram-scale.

### Hectogram-Scale

At room temperature and under the protection of nitrogen gas, 120.0 g of methylglycine ethyl ester hydrochloride A0, 458.4 g of ethyl formate, 120.0 g of anhydrous potassium carbonate, and 480 ml of 70% (V %) ethanol solution were added to a 3000 ml three-necked flask. After the addition, the reaction mixture was heated to 55-90° C. (in water bath) and reacted under stirring with a mechanical stirrer. The reaction was considered complete when HPLC showed that the sample contained less than 1.0% of the starting material A0 (the reaction duration lasted for about 15 hours). Subsequently, the reaction mixture was cooled to 25-30° C. and filtered: the collected filtrate was concentrated under reduced pressure at 40-45° C. to remove ethanol until no dripping: the residue was extracted with ethyl acetate twice, 300 ml each time; the extract liquors were combined, dried with anhydrous sodium sulfate and filtered. The collected filtrate was concentrated under reduced pressure at 40-45° C. until no dripping, to give 101 g of A1.

101 g of A1 obtained in the previous step was added to 150 ml of toluene, to which then 100 g of ethyl formate was added. The resulting solution was reacted in a 1000 ml three-necked flask, and then cooled to a temperature ranging from −5 to 5° C. (in ice-salt bath). Using a dropping funnel, 50 g of frozen sodium ethoxide (in ethanol solution) was added at a controlled rate to keep the reaction solution below a temperature of 5° C. After the addition, the mixture solution was reacted for 16 hours with the temperature being controlled in the range of from −5 to 5° C. until the reaction was complete. The reaction solution was poured into a separating funnel, then added with 1000 ml of petroleum ether, and stirred. The upper-layer supernatant was poured out, and the lower-layer liquid was collected.

The lower-layer liquid was transferred to a 3000 ml three-necked flask, added with 1000 ml of ethanol under stirring, and further added with 200 ml of concentrated hydrochloric acid (31% by mass). The resulting solution was heated to 60-80° C., reacted under reflux for 1.5 h, and then cooled to 25-30° C., and filtered. The collected filtrate was concentrated under reduced pressure at 40-45° C. to remove ethanol until no dripping, and then cooled to 25-30° C.

To the cooled reaction liquor of about 150 ml in volume, 1000 ml of acetic acid (glacial acetic acid), 52 g of cyanamide (in 50% aqueous solution), and 113 g of anhydrous sodium acetate were added. After the addition, the resulting solution was heated to 90-100° C., reacted under reflux for 2 hours, and then cooled to 20-25° C. The cooled reaction solution was adjusted with concentrated hydrochloric acid to a pH value of 1, and then concentrated under reduced at 45-50° C. to remove acetic acid until no dripping. The remaining solution was adjusted with potassium carbonate powder (in a slow and cautious addition speed and under stirring) to a pH value of 8-9, stirred for 1 hour and then filtered. The filtered cake was washed with water twice to give a solid, which was vacuum-dried under reduced pressure at 40° C.-50° C. to obtain 48.05 g of the product A4, with a purity of 87.58%.

### Kilogram-Scale

At room temperature and under the protection of nitrogen gas (with the same reaction process when nitrogen gas was used), 1900 g of methylglycine ethyl ester hydrochloride A0, 7258 g of ethyl formate, 1900 g of anhydrous potassium carbonate, and 7600 ml of 70% (V %) ethanol solution were added to a 25 L glass reactor kettle (with a heating or cooling liquid jacket). After the addition, the reaction mixture was heated to 55-90° C. (in water bath) and reacted under stirring with a mechanical stirrer. The reaction was considered complete when HPLC showed that the sample contained less than 1.0% of the starting material A0 (the reaction duration lasted for about 12 hours). Subsequently, the reaction mixture was cooled to 25-30° C. and filtered: the collected filtrate was concentrated under reduced pressure at 40-45° C. until no dripping: the residue was extracted with ethyl acetate five times, about 3 L each time: the extract liquors were combined, dried with anhydrous sodium sulfate and filtered. The collected filtrate was concentrated under reduced pressure at 40-45° C. until no dripping, to give 1706 g of A1.

1706 g of A1 obtained in the previous step was added to a 25 L glass reactor kettle (with a heating or cooling liquid jacket). The resulting solution was added with 2.5 L of toluene and 1750 g of ethyl formate, and then stirred. The reaction solution was cooled to −5 to 5° C. (in refrigeration cycle with ethanol as a heat exchange agent). Using a dropping funnel, 800 g of frozen sodium ethoxide (in ethanol solution) was added at a controlled rate to keep the reaction solution below a temperature of 5° C. After the addition, the mixture solution was reacted for 10 hours with the temperature being controlled at −5 to 5° C. until the reaction was complete. The reaction solution was transferred to a liquid-separating kettle, where it was then added with 4 L of petroleum ether and stirred, and after liquid separation, the lower-layer liquid was collected; this operation was repeated 4 times, and the collected and combined lower-layer liquids amounted to 4.5 L in total.

4.5 L of the lower-layer liquid was transferred to a 25 L glass reactor kettle (with a heating or cooling liquid jacket), added with 15 L of ethanol under stirring, and further added with 3.2 L of concentrated hydrochloric acid (31% by mass). The resulting solution was heated to 60-80° C., reacted under reflux for 1.5 h, and then cooled to 25-30° C., and filtered. The collected filtrate was concentrated under reduced pressure at 40-45° C. to remove ethanol until no dripping, and then cooled to 25-30° C.

The cooled reaction solution (still in the 25 L glass reactor kettle) was added with 15.7 L of acetic acid (glacial acetic acid), 820 g of cyanamide (50% aqueous solution), and 1780 g of anhydrous sodium acetate. After the addition, the resulting solution was heated to 90-100° C., reacted under reflux for 2 hours, and then cooled to 20-25° C. The cooled reaction solution was adjusted with concentrated hydrochloric acid to a pH value of 1, and then concentrated under reduced pressure at 45-50° C. to remove acetic acid until no dripping. The remaining solution was adjusted with potassium carbonate powder (in a slow and cautious addition speed and under stirring) to a pH value of 8-9, subjected to a more vigorous stirring (for about 1.5 hours) and then filtered. The filter cake was washed with water (5% ammonia water) twice to give a solid, which was vaccum-dried under reduced pressure at 40° C.-50° C. to obtain 1.26 kg of the product A4, with a purity of 73% and a yield of 60.3%.

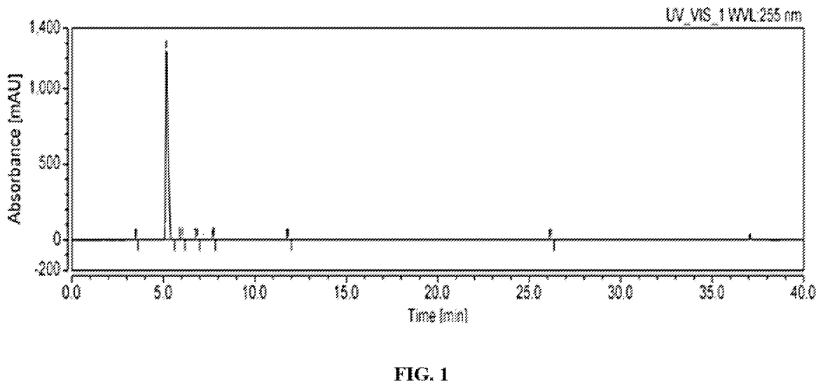

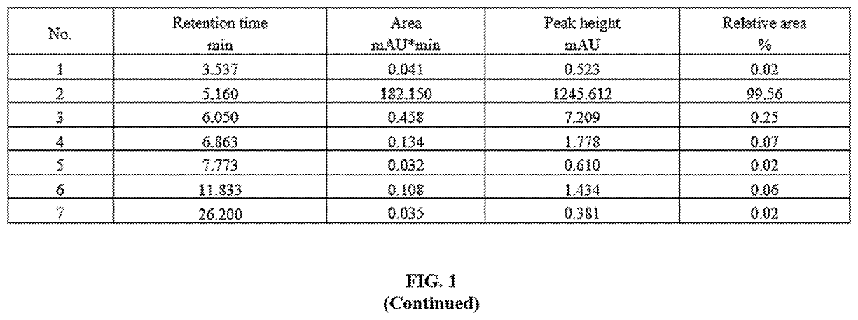

As the product of this batch had lower purity than that of the smaller-scale production, it was subjected to the following refining process:
The above crude product was dispersed into 1.1- to 1.5-fold volume of ethanol at 40-50° C., stirred slowly for 10-30 minutes, and filtered while it remained lukewarm. The filter cake was washed with cold 5% ammonia water three times, and then collected and had a purity of 85-91%. The ethanol and ammonia water used in the filtration were heated to 40-50° C. for redissolution and recrystallization. The crystals formed were collected, and had a purity of higher than 95%. FIG. 1 is the HPLC test result from the products of the kilogram-scale batch after recrystallization and purification, showing that these products had a purity of 99.56%.

### Pilot-Scale Batch

At room temperature and under the protection of nitrogen gas, 4300 g of methylglycine ethyl ester hydrochloride A0, 16426 g of ethyl formate, 4300 g of anhydrous potassium carbonate, and 17200 ml of 70% (V %) ethanol solution were added to a 50 L glass reactor kettle (with a heating or cooling liquid jacket). After the addition, the reaction mixture was heated to 55-90° C. (in water bath) and reacted under stirring with a mechanical stirrer. The reaction was considered complete when HPLC showed that the sample contained less than 1.0% of the starting material A0 (the reaction duration lasted for about 12 hours). Subsequently, the reaction mixture was cooled to 25-30° C. and filtered: the collected filtrate was concentrated under reduced pressure at 40-45° C. until no dripping: the residue was extracted with ethyl acetate 5 times, about 6 L each time: the extract liquors were combined, dried with anhydrous sodium sulfate and filtered. The filtrate was collected and concentrated under reduced pressure at 40-45° C. until no dripping, to give 4050 g of A1.

4000 g of A1 obtained in the previous step was added to a 50 L glass reactor kettle (with a heating or cooling liquid jacket). The resulting solution was added with 5.9 L of toluene and 4100 g of ethyl formate, and then stirred. The reaction solution was cooled to −5 to 5° C. (in refrigeration cycle with ethanol as a heat exchange agent). Using a dropping funnel, 1875 g of frozen sodium ethoxide (in ethanol solution) was added at a controlled rate to keep the the reaction solution below a temperature of 5° C. After the addition, the mixture solution was reacted for 10 hours with the temperature being controlled in the range of from −5 to 5° C. until the reaction was complete. The reaction solution was transferred to a liquid-separating kettle, where it was then added with 10 L of petroleum ether and stirred, and after liquid separation, the lower-layer liquid was collected; this operation was repeated 4 times, and the collected and combined lower-layer liquids amounted to 10.5 L in total.

10.5 L of the lower-layer liquid was transferred to a 50 L glass reactor kettle (with a heating or cooling liquid jacket), added with 30 L of ethanol under stirring, and further added with 7.5 L of concentrated hydrochloric acid (31% by mass). The resulting solution was heated to 60-80° C., reacted under reflux for 1.5 hours, and then cooled to 25-30° C., and filtered. The collected filtrate was concentrated under reduced pressure to remove ethanol at 40-45° C. until no dripping, and then cooled to 25-30° C.

The cooled reaction solution (still in the 50 L glass reactor kettle) was added with 37 L of acetic acid (glacial acetic acid), 1920 g of cyanamide (50% aqueous solution), and 4170 g of anhydrous sodium acetate. After the addition, the resulting solution was heated to 90-100° C., reacted under reflux for 2 hours, and then cooled to 20-25° C. The cooled reaction solution was adjusted with concentrated hydrochloric acid to a pH value of 1, and then concentrated under reduced pressure at 45-50° C. to remove acetic acid until no dripping. The residue was adjusted with potassium carbonate powder (in a slow and cautious addition speed and under stirring) to a pH value of 8-9, subjected to a more vigorous stirring (for about 1.5 hours) and then filtered. The filter cake was washed with water (5% ammonia water) twice to give a solid, which was vacuum-dried under reduced pressure at 40° C.-50° C. to obtain 3660 g of the product A4, with a purity of 98.2% and a yield of 77.2%.

### Example V

### Example 5 illustrates the method for detecting the purity of ethyl 1-N-methyl-2-amino-imidazole-5-carboxylate A4

The purified recrystallite of the ethyl 1-N-methyl-2-aminoimidazole-5-carboxylate A4 obtained from the kilogram-scale products of Example 4 was the sample to be tested, and HPLC was used for purity detection. The test samples were dissolved in 50% acetonitrile and then injected into an HPLC device for detection.

A reverse-phase C18 column was used as the separation chromatographic column, and the detection wavelength was 255 nm. The mobile phase A was 0.1% phosphoric acid solution, and the mobile phase B was acetonitrile. The gradient elution program included the mobile phase A from 95% volume ratio to 10% volume ratio, and gradient elution at 10% volume ratio for a period of time. 1-N-methyl-2-nitro-5-hydroxymethylimidazole A4 gave rise to a peak when the volume percentage of the mobile phase A was in the range of 70%-85%. The purity % of ethyl 1-N-methyl-2-aminoimidazole-5-carboxylate A4 was calculated by area normalization method based on its peak area.

Using a commercially available ZORBAX Eclipse Plus C18 column (specification of 150*4.6 mm, i.e., 150 mm in length and 4.6 mm in inner diameter) with 3.5 μm particle size filler as an example, and with the flow rate of the mobile phase being 1.0 m1/min, the elution procedure was as follows:

TABLE 2

| | Time/min. | | | | |
|---|---|---|---|---|---|
| | 0 | 30 | 35 | 35.1 | 40 |
| Mobile phase A % | 95 | 10 | 10 | 95 | 95 |
| Mobile phase B % | 5 | 90 | 90 | 5 | 5 |

As shown in FIG. 1, the peak retention time of ethyl 1-N-methyl-2-aminoimidazole-5-carboxylate A4 is at about 5.2 minutes. During the actual detection process, depending on the temperature and conditions of the instrument used, the peak retention time was in the range of 3-7 minutes.

The above-mentioned specific embodiments only represent the preferred ones of the present invention, while the protection scope of the present invention is not limited thereto. All the alternations or alternatives that could be easily contemplated by a skilled person familiar with the relevant technical field are covered within the technical scope of the present invention.

The invention claimed is:

1. A process for preparing an intermediate of aminoimidazole carboxylate, characterized in that
subjecting a compound of formula I-2 and a formate as the reactants to a first-step reaction in a benzene solvent and at a reaction temperature of −5° C.-5° C., with the addition of a solution of sodium alkoxide in such a way that the temperature of the reaction solution is not higher than 5° C., to obtain the intermediate of aminoimidazole carboxylate or a mixture comprising a compound of formula I-3, i.e., the intermediate of aminoimidazole carboxylate;
wherein the compounds of formulae I-2 and I-3 have the following structures, respectively: 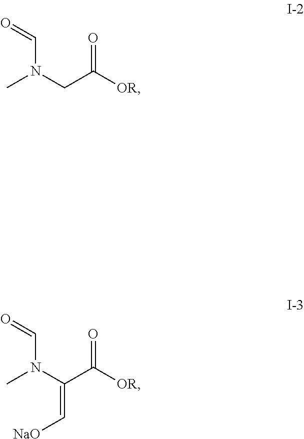
wherein the R group is a substituted or unsubstituted C₁-C₆ alkyl group.

2. The process for preparing the intermediate of aminoimidazole carboxylate according to claim 1, characterized in that
the benzene solvent is C₇-C₉ liquid-state substituted benzene, benzene or a mixture thereof;
the formate is methyl formate, ethyl formate, n-propyl formate, isopropyl formate or a mixture thereof;
the sodium alkoxide is selected from sodium methylate, sodium ethylate, sodium n-propoxide, or sodium isopropoxide;
the R group in the compounds of formulae I-2 and I-3 is a C₁-C₃ alkyl group;
the reaction temperature of the first-step reaction is ranging from −5 to 0° C.;
the reaction time of the first-step reaction is 8 to 16 hours;
in the first-step reaction, the volumetric mass ratio of the benzene solvent to the compound of formula I-2 is (1.40 m1-1.50 m1): 1 g.

3. The process for preparing the intermediate of aminoimidazole carboxylate according to claim 1, characterized in that
the mass ratio of the compound of formula I-2 to the sodium alkoxide is less than or equal to 1; and
the mass ratio of the compound of formula I-2 to the formate is less than or equal to 0.5.

4. The process for preparing the intermediate of aminoimidazole carboxylate according to claim 1, characterized in that the process further comprises the following steps:
adding, to the mixture obtained in the first-step reaction, a weak polar solvent in a volume 3-5 times as much as that of the mixture; after stratification, removing the upper layer to leave the lower layer as a reactant for an acid hydrolysis step, wherein the weak polar solvent is petroleum ether, diethyl ether or n-hexane.

5. A process for synthesizing aminoimidazole carboxylate, characterized in that the process comprises the following steps:
preparing a compound of formula I-3 (i.e., the intermediate of aminoimidazole carboxylate) or a mixture comprising the compound of formula I-3 (i.e., the intermediate of aminoimidazole carboxylate) through the process according to claim 1;
subjecting the compound of formula I-3 (i.e., the intermediate of aminoimidazole carboxylate) or a mixture comprising the compound of formula I-3 (i.e., the intermediate of aminoimidazole carboxylate) as the starting material, to acid hydrolysis to obtain a hydrolyzate of formula I-4;
wherein the hydrolyzate of formula I-4 is: 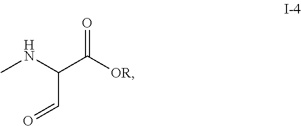
cyclizing the hydrolyzate of formula I-4 with cyanamide as reactant, to obtain a mixture comprising a compound of formula I, i.e., aminoimidazole carboxylate;
wherein, the compound of formula I is: 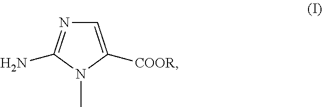
wherein the R group in the hydrolyzate of formula I-4 and in the compound of formula I is a C₁-C₆ alkyl group.

6. The process for synthesizing aminoimidazole carboxylate according to claim 5, characterized in that
the acid hydrolysis comprises the following steps:
adding under stirring, to the compound of formula I-3 (i.e., the intermediate of aminoimidazole carboxylate) or a mixture comprising the compound of formula I-3 (i.e., the intermediate of aminoimidazole carboxylate), ethanol or methanol in a volume 3-5 times as much as that of the compound or the mixture; adding concentrated hydrochloric acid; and then heating to 60-80° C., reacting under reflux for 0.5-2 hours and filtering; concentrating the collected filtrate under reduced pressure at 40-45° C. until no dripping, to obtain the hydrolyzate of formula I-4;
the cyclization reaction comprises the following steps:
mixing and dissolving the hydrolyzate of formula I-4 within an aqueous solution of cyanamide and an acetic acid solution of sodium acetate or a formic acid solution of sodium formate; heating and reacting under reflux to obtain a mixture comprising the compound of formula I, i.e., the aminoimidazole carboxylate.

7. The process for synthesizing aminoimidazole carboxylate according to claim 6, characterized in that
the mass ratio of cyanamide in the aqueous solution of cyanamide to the hydrolyzate of formula I-4 is 2:1;
the mass ratio of sodium acetate or sodium formate to the hydrolyzate of formula I-4 is 2:1; and
the volumetric mass ratio of the solvent acetic acid or formic acid to the hydrolyzate of formula I-4 is 10 ml: 1 g.

8. The process for synthesizing aminoimidazole carboxylate according to claim 5, characterized in that the process, after the cyclization reaction, further comprises the following steps:
adjusting, with hydrochloric acid or sulfuric acid, the reaction mixture solution comprising the compound of formula I, i.e., the aminoimidazole carboxylate, obtained after the cyclization reaction, to a pH of 1, and then after thermal removal of hydrochloric acid or sulfuric acid, adjusting with a base to a pH of 8 to 9, stirring and filtering; and washing the filter cake with water to give a solid, which is dried to obtain the compound of formula I, i.e., the aminoimidazole carboxylate.

9. An HPLC method for detecting the purity of ethyl 1-N-methyl-2-aminoimidazole-5-carboxylate, characterized in that a sample solution comprising ethyl 1-N-methyl-2-amino-imidazole-5-carboxylate is subjected to liquid chromatography under the following chromatographic conditions:
the separation column is a reverse-phase C18 column;
the detection wavelength is 255 nm;
mobile phase A is 0.1% phosphoric acid solution, and mobile phase B is acetonitrile;
elution is carried out using a gradient elution procedure; and
the purity % of ethyl 1-N-methyl-2-aminoimidazole-5-carboxylate is calculated by using the area normalization method based on its peak area.

10. The HPLC method for detecting the purity according to claim 9, characterized in that:
the gradient elution procedure is:

| | Time/min. | | | | |
|---|---|---|---|---|---|
| | 0 | 30 | 35 | 35.1 | 40 |
| Mobile phase A % | 95 | 10 | 10 | 95 | 95 |
| Mobile phase B % | 5 | 90 | 90 | 5 | 5 |

wherein % is volume-based; and
the peak retention time of the ethyl 1-N-methyl-2-aminoimidazole-5-carboxylate is in the range of from 3 to 7 minutes.

11. The process for preparing the intermediate of aminoimidazole carboxylate according to claim 2, characterized in that
the benzene solvent is toluene, xylene, trimethylbenzene, ethylbenzene, propylbenzene, isopropylbenzene or a mixture thereof.

12. The process for preparing the intermediate of aminoimidazole carboxylate according to claim 2, characterized in that
the R group in the compounds of formulae I-2 and I-3 is ethyl.

13. The process for preparing the intermediate of aminoimidazole carboxylate according to claim 3, characterized in that
the mass ratio of the compound of formula I-2 to the sodium alkoxide is in the range of from 0.80 to 1; and
the mass ratio of the compound of formula I-2 to the formate is in the range of from 0.4 to 0.5.

14. The process for synthesizing aminoimidazole carboxylate according to claim 5, characterized in that
the R group in the hydrolyzate of formula I-4 and in the compound of formula I is a C₁-C₃ alkyl group.

15. The process for synthesizing aminoimidazole carboxylate according to claim 14, characterized in that
the R group in the hydrolyzate of formula I-4 and in the compound of formula I is an ethyl group.
